(19) United States
(12) Patent Application Publication
(10) Pub. No.: US 2021/0241121 A1
(43) Pub. Date: Aug. 5, 2021

(54) Systems Configured for Cell-Based Histopathological Learning and Prediction and Methods Thereof

(71) Applicant: Origin Labs, Inc., San Francisco, CA (US)

(72) Inventors: Darick M. Tong, San Francisco, CA (US); Nishant Borude, San Francisco, CA (US); Nivedita Suresh, San Francisco, CA (US); Evan Szu, Zephyr Cove, NV (US); Clifford Szu, Zephyr Cove, NV (US)

(21) Appl. No.: 17/168,791

(22) Filed: Feb. 5, 2021

Related U.S. Application Data
(60) Provisional application No. 62/970,338, filed on Feb. 5, 2020.

(51) Int. Cl. G06N 3/08; G06N 3/04

(52) U.S. Cl. CPC G06N 3/088; G06N 3/0454

(57) ABSTRACT

Histopathological scoring can be based on ratios of different types of cells, and in particular, cells which exhibit a particular genotypic or phenotypic characteristic, as identified by a biological assay. Automating the scoring process with an image analysis algorithm requires both correctly delineating cells, a process known as segmentation, and classifying each cell according to its morphology and reactivity to the assay. Successful classification thus depends on both successful segmentation and successful classification, resulting in the error rates of the two steps being compounded. Systems and methods of the present disclosure reduce error by performing the cell counting and classification task in a single step using a generative adversarial network (or GAN). The present disclosure similarly employs a GAN for counting cells by representing the training data as a Gaussian at the center of each cell nucleus.

### RELATED APPLICATIONS

This application claims priority to and the benefit of U.S. Provisional Patent Application No. 62/970,338, filed Feb. 5, 2020, which is incorporated by reference herein in its entirety.

### COPYRIGHT NOTICE

A portion of the disclosure of this patent document contains material that is subject to copyright protection. The copyright owner has no objection to the facsimile reproduction by anyone of the patent document or the patent disclosure, as it appears in the Patent and Trademark Office patent files or records, but otherwise reserves all copyright rights whatsoever. The following notice applies to the software and data as described below and in drawings that form a part of this document: Copyright, Origin Labs, All Rights Reserved.

### FIELD OF TECHNOLOGY

The present disclosure generally relates to computer-based systems, devices and components configured for one or more novel technological applications of cell-based histopathological learning and prediction and methods thereof, e.g., using cell, cell culture, tissue or other imagery.

### BACKGROUND OF TECHNOLOGY

Histopathological scoring can be based on ratios of different types of cells, and in particular, cells which exhibit a particular genotypic or phenotypic characteristic, as identified by a biological assay. Scoring is generally done by a medical expert, who analyzes a tissue sample stained with the appropriate assay and estimates the ratio of the cells of interest.

### BRIEF DESCRIPTION OF THE DRAWINGS

Various embodiments of the present disclosure can be further explained with reference to the attached drawings, wherein like structures are referred to by like numerals throughout the several views. The drawings shown are not necessarily to scale, with emphasis instead generally being placed upon illustrating the principles of the present disclosure. Therefore, specific structural and functional details disclosed herein are not to be interpreted as limiting, but merely as a representative basis for teaching one skilled in the art to variously employ one or more illustrative embodiments.

FIG. 1 depicts a block diagram of an exemplary system for automated cell-based histopathological scoring using a histopathological score model according to one or more embodiments of the present disclosure.

FIG. 2 depicts a block diagram of an exemplary architecture for automated cell-based histopathological scoring using a generative adversarial network according to one or more embodiments of the present disclosure.

FIG. 3 depicts a block diagram of an exemplary architecture for training a generative adversarial network to predict automated cell-based histopathological scoring including training a generative adversarial network according to one or more embodiments of the present disclosure.

FIG. 4 depicts a block diagram of an exemplary architecture for a generative adversarial network for predicting automated cell-based histopathological scoring according to one or more embodiments of the present disclosure.

FIG. 5 depicts a block diagram of an exemplary computer-based system and platform in accordance with one or more embodiments of the present disclosure.

FIG. 6 depicts a block diagram of another exemplary computer-based system and platform in accordance with one or more embodiments of the present disclosure.

FIG. 7 illustrates schematics of an exemplary implementation of the cloud computing/architecture(s) in which the illustrative computer-based systems or platforms of the present disclosure may be specifically configured to operate.

FIG. 8 illustrates schematics of another exemplary implementation of the cloud computing/architecture(s) in which the illustrative computer-based systems or platforms of the present disclosure may be specifically configured to operate

FIG. 9 depicts an illustrative segmentation and classification of cells for cell-based scoring according to aspects of embodiments of the present disclosure.

### DETAILED DESCRIPTION

Various detailed embodiments of the present disclosure, taken in conjunction with the accompanying figures, are disclosed herein; however, it is to be understood that the disclosed embodiments are merely illustrative. In addition, each of the examples given in connection with the various embodiments of the present disclosure is intended to be illustrative, and not restrictive.

Throughout the specification, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments may be readily combined, without departing from the scope or spirit of the present disclosure.

In addition, the term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

As used herein, the terms “and” and “or” may be used interchangeably to refer to a set of items in both the conjunctive and disjunctive in order to encompass the full description of combinations and alternatives of the items. By way of example, a set of items may be listed with the disjunctive “or”, or with the conjunction “and.” In either case, the set is to be interpreted as meaning each of the items singularly as alternatives, as well as any combination of the listed items.

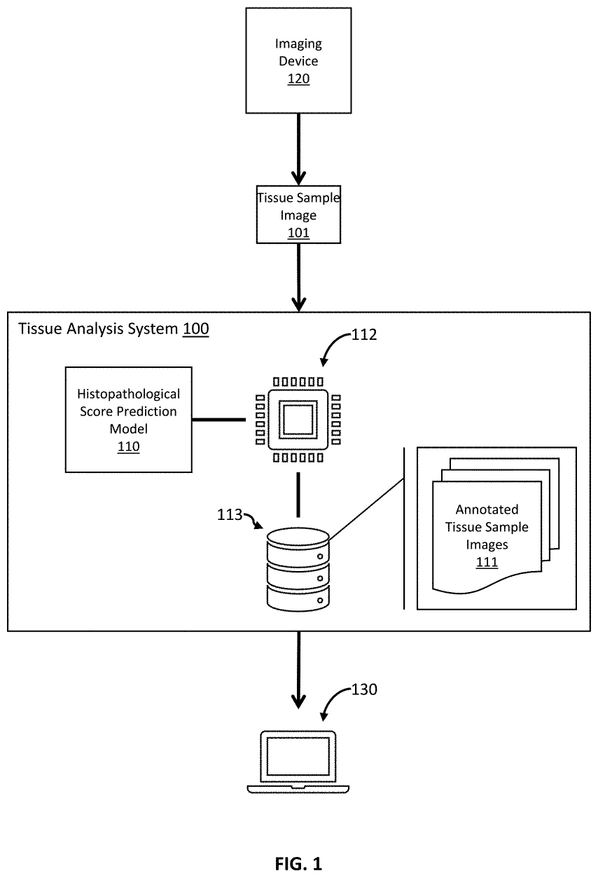

FIGS. 1 through 5 illustrate systems and methods of histopathological scoring using imagery of cells, such as in tissues or other cell cultures. Automating the scoring process with an image analysis algorithm requires both correctly delineating cells, a process known as segmentation, and classifying each cell according to its morphology and reactivity to the assay. Successful classification thus depends on both successful segmentation and successful classification; in other words, the error rates of the two steps are compounded. The following embodiments provide technical solutions and technical improvements that overcome technical problems, drawbacks and/or deficiencies in the technical fields involving accurately and efficiently delineating cells and categorizing the cells according to morphology and reactivity to an assay. As explained in more detail, below, technical solutions and technical improvements herein include aspects of improved delineating and categorization by automatically accomplishing the cell counting and classification task in a single step using a generative adversarial network (or GAN). GANs have often been used for “crowd counting” or estimating the number of people in a photograph of a crowd. The presently disclosed embodiments can employ a GAN for counting cells by representing the training data as a Gaussian at the centered at a center of each cell nucleus. Based on such technical features, further technical benefits become available to users and operators of these systems and methods. Moreover, various practical applications of the disclosed technology are also described, which provide further practical benefits to users and operators that are also new and useful improvements in the art.

FIG. 1 depicts a block diagram of an exemplary system for automated cell-based histopathological scoring using a histopathological score model according to one or more embodiments of the present disclosure.

In some embodiments, a tissue analysis system 100 may ingest tissue sample images 101 from, e.g., one or more imaging devices 120. In some embodiments, the imaging device 120 may include, e.g., a digital microscope, an electron microscope, a digital camera, or any other device suitable for imaging cells of a tissue sample.

In some embodiments, the imaging device 120 is in communication with the tissue analysis system 100. In some embodiments, the imaging device 120 may be connected to the tissue analysis system 100 via a physical interface, such as, e.g., a bus, Universal Serial Bus (USB), serial ATA (SATA), Peripheral Component Interconnect (PCI), Peripheral Component Interconnect Express (PCIe), non-volatile memory express (NVME), Ethernet, or any other suitable wired data communication solution.

In some embodiments, the imaging device 120 may communicate the tissue sample images 101 over a wireless connection, e.g., over one or more suitable data communication networks (e.g., the Internet, satellite, etc.) and utilizing one or more suitable data communication protocols/modes such as, without limitation, IPX/SPX, X.25, AX.25, AppleTalk™, TCP/IP (e.g., HTTP), Bluetooth™, near-field wireless communication (NFC), RFID, Narrow Band Internet of Things (NBIOT), 3G, 4G, 5G, GSM, GPRS, WiFi, WiMax, CDMA, satellite, ZigBee, and other suitable communication modes.

In some embodiments, the tissue analysis system 100 may be a part of a computing device, such as, e.g., a laptop computer, a desktop computer, a mobile device (e.g., a smartphone, tablet or wearable device), a server, a cloud computing system, or any other suitable computer device or any combination thereof. Thus, the tissue analysis system 100 may include hardware components such as a processor 112, which may include local or remote processing components. In some embodiments, the processor 112 may include any type of data processing capacity, such as a hardware logic circuit, for example, an application specific integrated circuit (ASIC) and a programmable logic, or such as a computing device, for example, a microcomputer or microcontroller that include a programmable microprocessor. In some embodiments, the processor 112 may include data-processing capacity provided by the microprocessor. In some embodiments, the microprocessor may include memory, processing, interface resources, controllers, and counters. In some embodiments, the microprocessor may also include one or more programs stored in memory.

Similarly, the tissue analysis system 100 may include storage 113, such as local hard-drive, solid-state drive, flash drive, database or other local storage, or remote storage such as a server, mainframe, database or cloud provided storage solution.

In some embodiments, the storage 113 may store data related to histopathological scoring of the tissue sample image 101. For example, the storage 113 may store the tissue sample image 101 before, during or after tissue analysis and histopathological score prediction, or any combination thereof. The storage 113 may also or instead store annotated tissue sample images 111, e.g., of the type of tissue represented in the tissue sample image 101. The annotated tissue sample images 111 may be accessed via the storage 113 by the processor 112 such that system components (e.g., the histopathological score prediction model 110) may be trained to classify and count cells of each cell type to generate a histopathological score prediction.

In some embodiments, the tissue analysis system 100 may implement computer engines for histopathological scoring prediction for the tissues represented in the tissue sample image 101, such as, e.g., the histopathological score prediction model 110. In some embodiments, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Examples of software may include software components, programs, applications, computer programs, application programs, system programs, machine programs, operating system software, middleware, firmware, software modules, routines, subroutines, functions, methods, procedures, software interfaces, application program interfaces (API), instruction sets, computing code, computer code, code segments, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

In some embodiments, the histopathological score prediction model 110 may include dedicated and/or shared software components, hardware components, or a combination thereof. For example, the histopathological score prediction model 110 may include a dedicated processor and storage, or may share hardware resources, including the processor 112 and storage 113 of the tissue analysis system 100, or any combination thereof. In some embodiments, the software and/or hardware components may be employed to execute functions of the histopathological score prediction model 110 to train the histopathological score prediction model 110 with the annotated tissue sample images 111, generate histopathological scores for the tissue sample image 101, among other functionality as is described in further detail below.

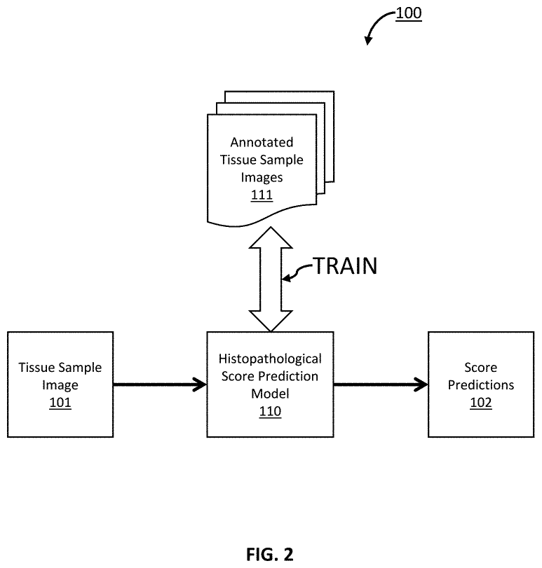

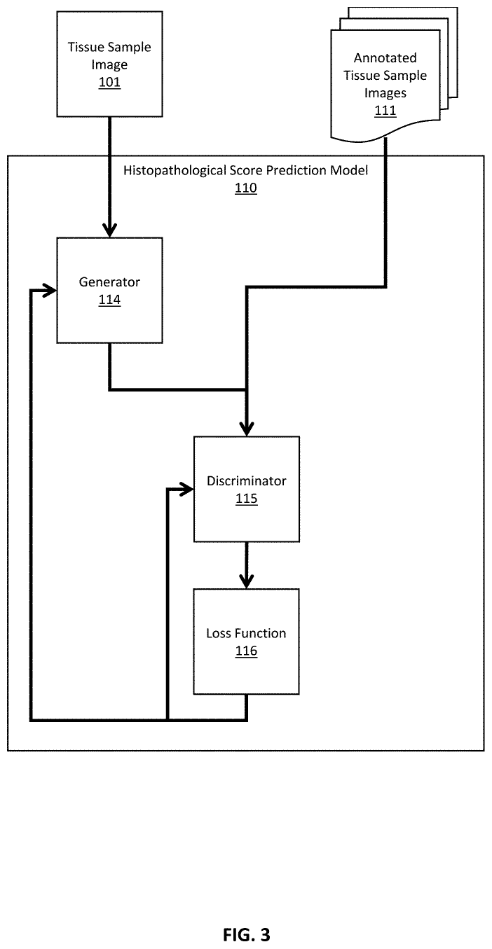

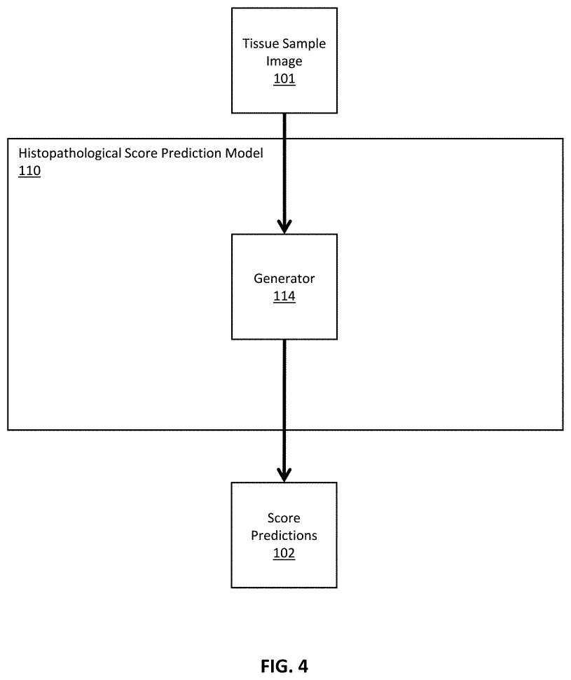

FIG. 2, FIG. 3 and FIG. 4 depict block diagrams of an exemplary architecture for automated cell-based histopathological scoring using a generative adversarial network according to one or more embodiments of the present disclosure.

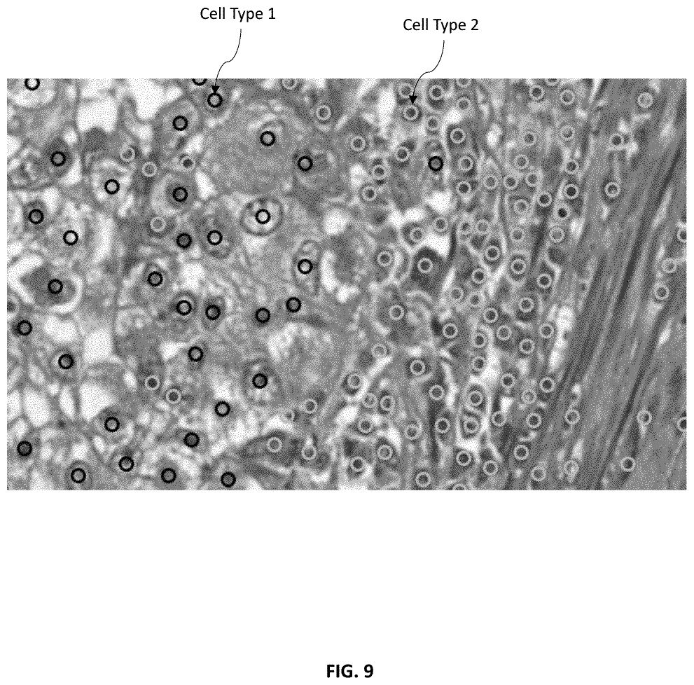

In some embodiments, a histopathological prediction model 110 predicts histopathological score predictions 102 for cells in a tissue sample image 101 based on training with annotated tissue sample images 111. In some embodiments, the histopathological prediction model 110 combines counting with classification by using a separate image band for each class of cell (see, for example FIG. 9). For example, a cell-counting algorithm which classifies cells into three types (e.g. Tumor, Immune, Stromal), would use three separate channels for three Gaussian masks for each cell, depending on its type. For example, classification into 4 types, may use 4 separate images (grayscale) for each cell type (all having Gaussians centered at each cell). Classification problems with more cell types would use images with the equivalent number of bands (there is no limit to the number of bands an image can encode for each pixel).

In some embodiments, any type of mask may be employed to identify and count the cells of a tissue sample image. The use of Gaussian masks algorithmically speeds up cell counting (sum of each Gaussian mask is 1, this gives cell counts according to a sum pixels in a mask). Moreover, the size of the Gaussian mask may be controlled, e.g., by varying parameters of expected value (0 and variance (a) of the Gaussian function. This helps to use bigger masks when the cells are bigger and far apart and smaller masks when they are smaller and closer to each other.

One advantage of training and using a model to identify and classify multiple cells at once is that the model learns contextual features of the histology, which are often important signals for correctly identifying visually similar cells. In some embodiments, the contextual features may include, e.g., cell morphology (e.g., tumor morphology in the case of tumor cells), neighboring cells, relative positioning of the cells with respect to the boundary of the region, distance from a different region, or other suitable feature of the histology or any combination thereof.

In some embodiments, the histopathological prediction model 110 may be configured to utilize one or more exemplary AI or machine learning techniques chosen from, but not limited to, decision trees, boosting, support-vector machines, neural networks, nearest neighbor algorithms, Naive Bayes, bagging, random forests, and the like. In some embodiments and, optionally, in combination of any embodiment described above or below, an exemplary neural network technique may be one of, without limitation, feedforward neural network, radial basis function network, recurrent neural network, convolutional network (e.g., U-net) or other suitable network. In some embodiments and, optionally, in combination of any embodiment described above or below, an exemplary implementation of neural network may be executed as follows:
i) define neural network architecture/model,
ii) transfer the input data to the exemplary neural network model,
iii) train the exemplary model incrementally,
iv) determine the accuracy for a specific number of timesteps,
v) apply the exemplary trained model to process the newly-received input data,
vi) optionally and in parallel, continue to train the exemplary trained model with a predetermined periodicity.

In some embodiments and, optionally, in combination of any embodiment described above or below, the exemplary trained neural network model may specify a neural network by at least a neural network topology, a series of activation functions, and connection weights. For example, the topology of a neural network may include a configuration of nodes of the neural network and connections between such nodes. In some embodiments and, optionally, in combination of any embodiment described above or below, the exemplary trained neural network model may also be specified to include other parameters, including but not limited to, bias values, functions and aggregation functions. For example, an activation function of a node may be a step function, sine function, continuous or piecewise linear function, sigmoid function, hyperbolic tangent function, or other type of mathematical function that represents a threshold at which the node is activated. In some embodiments and, optionally, in combination of any embodiment described above or below, the exemplary aggregation function may be a mathematical function that combines (e.g., sum, product, etc.) input signals to the node. In some embodiments and, optionally, in combination of any embodiment described above or below, an output of the exemplary aggregation function may be used as input to the exemplary activation function. In some embodiments and, optionally, in combination of any embodiment described above or below, the bias may be a constant value or function that may be used by the aggregation function and/or the activation function to make the node more or less likely to be activated.

In some embodiments, the histopathological score prediction model 110 utilizes a generative adversarial network (GAN) to generate the histopathological score predictions 102. To do so, the histopathological score prediction model 110 may be trained using the annotated tissue sample images 111. In some embodiments, medical experts annotate digital representations of tissue samples by placing an appropriately labeled (i.e. colored) annotation marking the center of each nucleus to form the annotated tissue sample images 111 establishing true cell type classifications for a training dataset. These annotations in the annotated tissue sample images 111 are converted to blank images with Gaussian distributions of pixel values at each pixel of the appropriate band (corresponding to each cell's label) centered at the center of each nucleus in the original image. The values of all pixels of each Gaussian sum to 1. In some embodiments, the histopathological score prediction model 110 is trained to generate the mask representing the segmentation and classification with Gaussians from the original unlabeled image (e.g., a training Gaussian mask for an annotated tissue sample image 111).

In some embodiments, GAN includes a generator 114 and a discriminator 115. In some embodiment, the generator 114 can be chosen among any suitable generator algorithm, such as, e.g., DenseNet, FCN, UNet or other architecture depending on the assay. In some embodiments, the discriminator 115 may include a suitable convolutional neural network, e.g., a similar network or similar type of network to the generator 114, such as, e.g., a ResNet, FCN, among others a relatively simple convolutional neural network.

In some embodiments, the generator 114 may generate separate masks for each cell type to mask cell locations, e.g., with a Gaussian mask. The discriminator 115 network is a classifier that identifies which mask is real (ground truth) or produced by generator 114. Accordingly, in some embodiments, the mask output by the generator 114 is compared with a real mask (e.g., the training Gaussian mask) using mean squared error (MSE), mean absolute error (MAE) or other suitable regression loss function 116. In some embodiments, the training is done in two stages to improve prediction accuracy: Pretraining just the generator 114 and training the complete GAN. Pretraining may utilize, e.g., 500 to 1000 epochs of training data or more, and training the GAN may utilize, e.g., up to 1000 epochs or more of training data.

In some embodiments, the training data includes the annotated images. Annotations may be converted into Gaussian masks that serve as ground truth training data for the GAN. The training Gaussian masks may be creating using, e.g., a bivariate normal function with a variance parameter σ centered at each cell (with μx and μy being the x,y coordinate of the cell center according to the expected value parameter μ) to create the training Gaussian mask.

In some embodiments, the GAN may automatically count the numbers of cells by estimating a sum of the mask produced by the generator after training. As a result, the GAN may output the sum of the output mask to also output the cell count.

In some embodiments, upon training, the histopathological score prediction model 110 may ingest each tissue sample image 101 and process a region of interest to produce an image with Gaussians. The values of all pixels are summed for each image band. The final sums of each band represent the counts of cells of the corresponding type. The histopathological score prediction 102 is computed using the resulting cell counts.

In some embodiments, the histopathological score prediction 102 may be computed using ratios of the counts of relevant cell types. For example, upon producing a cell count of each cell type from individual masks, a metric may be formed depending on the type of the assay, such as, e.g., a ratio of tumor cells to total cells, immune cells to total cells, or any other metric characterizing the prevalence of a particular cell type.

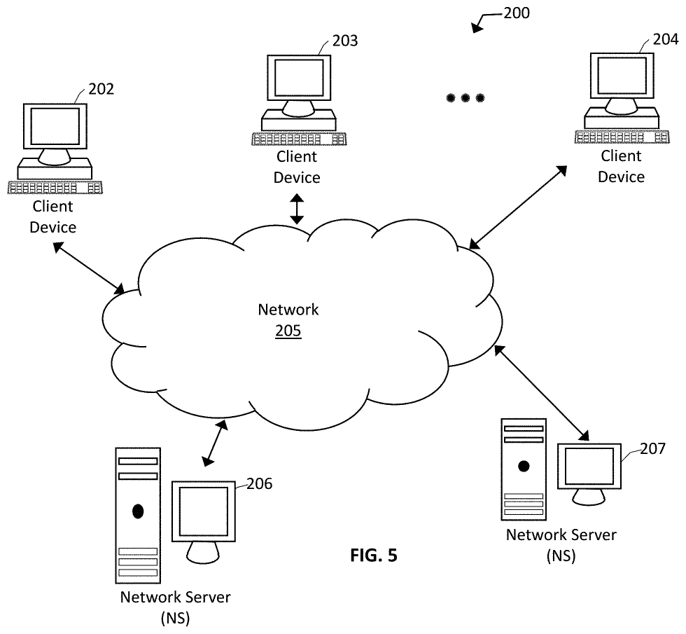

In some embodiments, the histopathological score prediction 102 may then be displayed to a user, such as, e.g., a patient care provider, a laboratory technician, or other professional, e.g., for diagnostic or study result data. For example, the histopathological score prediction 102 may be displayed, e.g., at a computing device 130 such as, e.g., a laptop computer, desktop computer, mobile device, thin client, terminal, etc., and/or at a client device 202-204 of FIG. 5 described below. Accordingly, a tissue sample or other cell imagery may be analyzed with test results including the histopathological score prediction 102 automatically generated by forming both a segmentation and a classification of the cells in the imagery and provided to a user quickly and efficient. By forming both a segmentation and a classification in a single step using the histopathological score prediction model 110, processing resources are reduced by reducing operations and memory required to analyze the imagery to improve both computational efficiency and imaging sophistication and accuracy. Thus, the fields and technologies of cellular imaging systems and image analysis systems are improved to more efficiently and accurate produce histopathological scores without user input.

FIG. 5 depicts a block diagram of an exemplary computer-based system and platform 200 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the illustrative computing devices and the illustrative computing components of the exemplary computer-based system and platform 200 may be configured to manage a large number of members and concurrent transactions, as detailed herein. In some embodiments, the exemplary computer-based system and platform 200 may be based on a scalable computer and network architecture that incorporates varies strategies for assessing the data, caching, searching, and/or database connection pooling. An example of the scalable architecture is an architecture that is capable of operating multiple servers.

In some embodiments, referring to FIG. 5, members 202-204 (e.g., clients) of the exemplary computer-based system and platform 200 may include virtually any computing device capable of receiving and sending a message over a network (e.g., cloud network), such as network 205, to and from another computing device, such as servers 206 and 207, each other, and the like. In some embodiments, the member devices 202-204 may be personal computers, multiprocessor systems, microprocessor-based or programmable consumer electronics, network PCs, and the like. In some embodiments, one or more member devices within member devices 202-204 may include computing devices that typically connect using a wireless communications medium such as cell phones, smart phones, pagers, walkie talkies, radio frequency (RF) devices, infrared (IR) devices, CBs, integrated devices combining one or more of the preceding devices, or virtually any mobile computing device, and the like. In some embodiments, one or more member devices within member devices 202-204 may be devices that are capable of connecting using a wired or wireless communication medium such as a PDA, POCKET PC, wearable computer, a laptop, tablet, desktop computer, a netbook, a video game device, a pager, a smart phone, an ultra-mobile personal computer (UMPC), and/or any other device that is equipped to communicate over a wired and/or wireless communication medium (e.g., NFC, RFID, NBIOT, 3G, 4G, 5G, GSM, GPRS, WiFi, WiMax, CDMA, satellite, ZigBee, etc.). In some embodiments, one or more member devices within member devices 202-204 may run one or more applications, such as Internet browsers, mobile applications, voice calls, video games, videoconferencing, and email, among others. In some embodiments, one or more member devices within member devices 202-204 may be configured to receive and to send web pages, and the like. In some embodiments, an exemplary specifically programmed browser application of the present disclosure may be configured to receive and display graphics, text, multimedia, and the like, employing virtually any web based language, including, but not limited to Standard Generalized Markup Language (SMGL), such as HyperText Markup Language (HTML), a wireless application protocol (WAP), a Handheld Device Markup Language (HDML), such as Wireless Markup Language (WML), WMLScript, XML, JavaScript, and the like. In some embodiments, a member device within member devices 202-204 may be specifically programmed by either Java, .Net, QT, C, C++ and/or other suitable programming language. In some embodiments, one or more member devices within member devices 202-204 may be specifically programmed to include or execute an application to perform a variety of possible tasks, such as, without limitation, messaging functionality, browsing, searching, playing, streaming or displaying various forms of content, including locally stored or uploaded messages, images and/or video, and/or games.

In some embodiments, the exemplary network 205 may provide network access, data transport and/or other services to any computing device coupled to it. In some embodiments, the exemplary network 205 may include and implement at least one specialized network architecture that may be based at least in part on one or more standards set by, for example, without limitation, Global System for Mobile communication (GSM) Association, the Internet Engineering Task Force (IETF), and the Worldwide Interoperability for Microwave Access (WiMAX) forum. In some embodiments, the exemplary network 205 may implement one or more of a GSM architecture, a General Packet Radio Service (GPRS) architecture, a Universal Mobile Telecommunications System (UMTS) architecture, and an evolution of UMTS referred to as Long Term Evolution (LTE). In some embodiments, the exemplary network 205 may include and implement, as an alternative or in conjunction with one or more of the above, a WiMAX architecture defined by the WiMAX forum. In some embodiments and, optionally, in combination of any embodiment described above or below, the exemplary network 205 may also include, for instance, at least one of a local area network (LAN), a wide area network (WAN), the Internet, a virtual LAN (VLAN), an enterprise LAN, a layer 3 virtual private network (VPN), an enterprise IP network, or any combination thereof. In some embodiments and, optionally, in combination of any embodiment described above or below, at least one computer network communication over the exemplary network 205 may be transmitted based at least in part on one of more communication modes such as but not limited to: NFC, RFID, Narrow Band Internet of Things (NBIOT), ZigBee, 3G, 4G, 5G, GSM, GPRS, WiFi, WiMax, CDMA, satellite and any combination thereof. In some embodiments, the exemplary network 205 may also include mass storage, such as network attached storage (NAS), a storage area network (SAN), a content delivery network (CDN) or other forms of computer or machine readable media.

In some embodiments, the exemplary server 206 or the exemplary server 207 may be a web server (or a series of servers) running a network operating system, examples of which may include but are not limited to Microsoft Windows Server, Novell NetWare, or Linux. In some embodiments, the exemplary server 206 or the exemplary server 207 may be used for and/or provide cloud and/or network computing. Although not shown in FIG. 5, in some embodiments, the exemplary server 206 or the exemplary server 207 may have connections to external systems like email, SMS messaging, text messaging, ad content providers, etc. Any of the features of the exemplary server 206 may be also implemented in the exemplary server 207 and vice versa.

In some embodiments, one or more of the exemplary servers 206 and 207 may be specifically programmed to perform, in non-limiting example, as authentication servers, search servers, email servers, social networking services servers, SMS servers, IM servers, MMS servers, exchange servers, photo-sharing services servers, advertisement providing servers, financial/banking-related services servers, travel services servers, or any similarly suitable service-base servers for users of the member computing devices 202-204.

In some embodiments and, optionally, in combination of any embodiment described above or below, for example, one or more exemplary computing member devices 202-204, the exemplary server 206, and/or the exemplary server 207 may include a specifically programmed software module that may be configured to send, process, and receive information using a scripting language, a remote procedure call, an email, a tweet, Short Message Service (SMS), Multimedia Message Service (MMS), instant messaging (IM), internet relay chat (IRC), mIRC, Jabber, an application programming interface, Simple Object Access Protocol (SOAP) methods, Common Object Request Broker Architecture (CORBA), HTTP (Hypertext Transfer Protocol), REST (Representational State Transfer), or any combination thereof.

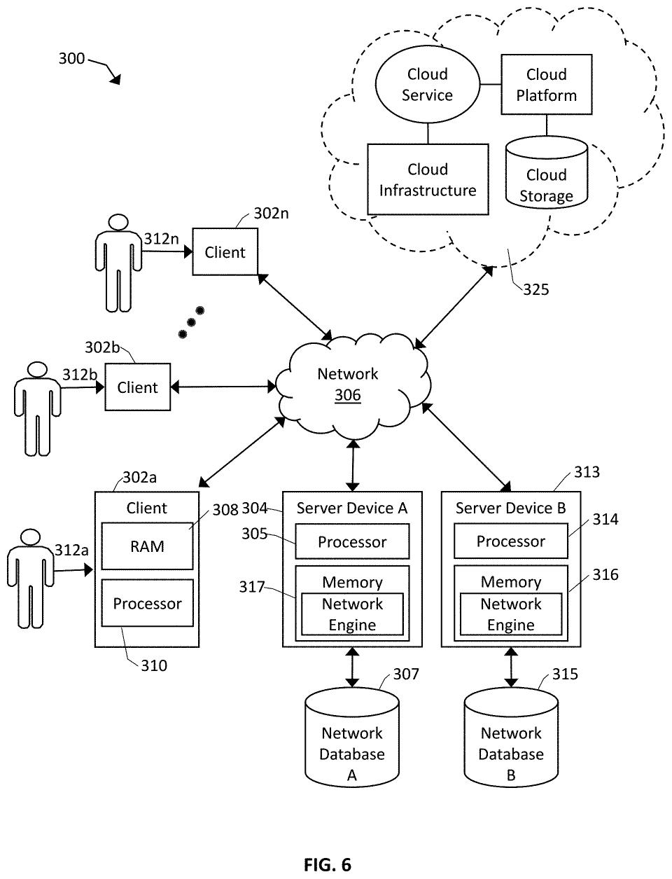

FIG. 6 depicts a block diagram of another exemplary computer-based system and platform 300 in accordance with one or more embodiments of the present disclosure. However, not all of these components may be required to practice one or more embodiments, and variations in the arrangement and type of the components may be made without departing from the spirit or scope of various embodiments of the present disclosure. In some embodiments, the member computing devices 302a, 302b through 302n shown each at least includes a computer-readable medium, such as a random-access memory (RAM) 308 coupled to a processor 310 or FLASH memory. In some embodiments, the processor 310 may execute computer-executable program instructions stored in memory 308. In some embodiments, the processor 310 may include a microprocessor, an ASIC, and/or a state machine. In some embodiments, the processor 310 may include, or may be in communication with, media, for example computer-readable media, which stores instructions that, when executed by the processor 310, may cause the processor 310 to perform one or more steps described herein. In some embodiments, examples of computer-readable media may include, but are not limited to, an electronic, optical, magnetic, or other storage or transmission device capable of providing a processor, such as the processor 310 of member computing device 302a, with computer-readable instructions. In some embodiments, other examples of suitable media may include, but are not limited to, a floppy disk, CD-ROM, DVD, magnetic disk, memory chip, ROM, RAM, an ASIC, a configured processor, all optical media, all magnetic tape or other magnetic media, or any other medium from which a computer processor can read instructions. Also, various other forms of computer-readable media may transmit or carry instructions to a computer, including a router, private or public network, or other transmission device or channel, both wired and wireless. In some embodiments, the instructions may comprise code from any computer-programming language, including, for example, C, C++, Visual Basic, Java, Python, Perl, JavaScript, etc.

In some embodiments, member computing devices 302a through 302n may also comprise a number of external or internal devices such as a mouse, a CD-ROM, DVD, a physical or virtual keyboard, a display, or other input or output devices. In some embodiments, examples of member computing devices 302a through 302n (e.g., clients) may be any type of processor-based platforms that are connected to a network 306 such as, without limitation, personal computers, digital assistants, personal digital assistants, smart phones, pagers, digital tablets, laptop computers, Internet appliances, and other processor-based devices. In some embodiments, member computing devices 302a through 302n may be specifically programmed with one or more application programs in accordance with one or more principles/methodologies detailed herein. In some embodiments, member computing devices 302a through 302n may operate on any operating system capable of supporting a browser or browser-enabled application, such as Microsoft™ Windows™, and/or Linux. In some embodiments, member computing devices 302a through 302n shown may include, for example, personal computers executing a browser application program such as Microsoft Corporation's Internet Explorer™, Apple Computer, Inc.'s Safari™, Mozilla Firefox, and/or Opera. In some embodiments, through the member computer devices 302a through 302n, users, 312a through 312n, may communicate over the exemplary network 306 with each other and/or with other systems and/or devices coupled to the network 306. As shown in FIG. 6, exemplary server devices 304 and 313 may be also coupled to the network 306. In some embodiments, one or more member computing devices 302a through 302n may be mobile clients.

In some embodiments, at least one database of exemplary databases 307 and 315 may be any type of database, including a database managed by a database management system (DBMS). In some embodiments, an exemplary DBMS-managed database may be specifically programmed as an engine that controls organization, storage, management, and/or retrieval of data in the respective database. In some embodiments, the exemplary DBMS-managed database may be specifically programmed to provide the ability to query, backup and replicate, enforce rules, provide security, compute, perform change and access logging, and/or automate optimization. In some embodiments, the exemplary DBMS-managed database may be chosen from Oracle database, IBM DB2, Adaptive Server Enterprise, FileMaker, Microsoft Access, Microsoft SQL Server, MySQL, PostgreSQL, and a NoSQL implementation. In some embodiments, the exemplary DBMS-managed database may be specifically programmed to define each respective schema of each database in the exemplary DBMS, according to a particular database model of the present disclosure which may include a hierarchical model, network model, relational model, object model, or some other suitable organization that may result in one or more applicable data structures that may include fields, records, files, and/or objects. In some embodiments, the exemplary DBMS-managed database may be specifically programmed to include metadata about the data that is stored.

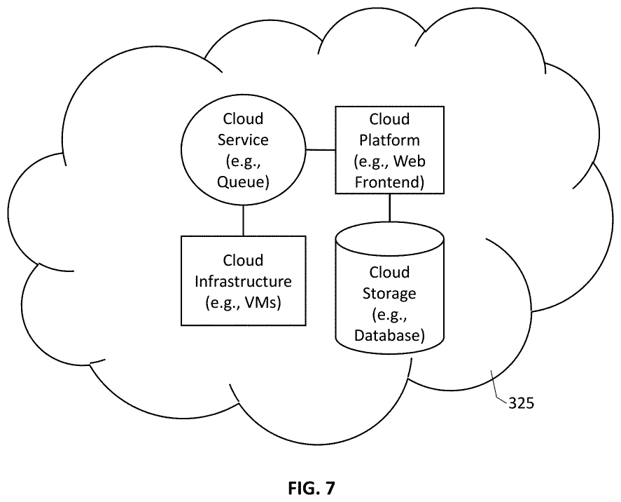

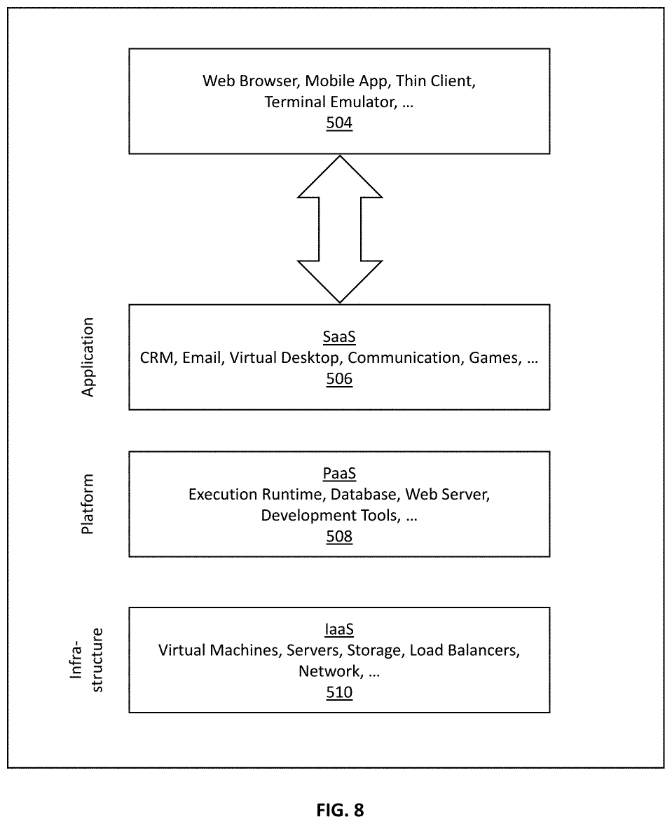

In some embodiments, the illustrative computer-based systems or platforms of the present disclosure may be specifically configured to operate in a cloud computing/architecture such as, but not limiting to: infrastructure a service (IaaS), platform as a service (PaaS), and/or software as a service (SaaS). FIG. 7 and FIG. 8 illustrate schematics of exemplary implementations of the cloud computing/architecture(s) in which the illustrative computer-based systems or platforms of the present disclosure may be specifically configured to operate.

FIG. 9 depicts an example labeled tissue sample image 101 as a result of the histopathological score prediction model 110. In this case, two types of cells (of Cell Type 1 and Cell Type 2) were identified and labeled using a Gaussian mask as described above. Because labelling is performed using Gaussian masks, the count of each of Cell Type 1 and Cell Type 2 can be extracted as a characteristic of the image according to the sum of each respective mask. The histopathological score may be determined according to a ratio of the counts.

It is understood that at least one aspect/functionality of various embodiments described herein can be performed in real-time and/or dynamically. As used herein, the term “real-time” is directed to an event/action that can occur instantaneously or almost instantaneously in time when another event/action has occurred. For example, the “real-time processing,” “real-time computation,” and “real-time execution” all pertain to the performance of a computation during the actual time that the related physical process (e.g., a user interacting with an application on a mobile device) occurs, in order that results of the computation can be used in guiding the physical process.

As used herein, the term “dynamically” and term “automatically,” and their logical and/or linguistic relatives and/or derivatives, mean that certain events and/or actions can be triggered and/or occur without any human intervention. In some embodiments, events and/or actions in accordance with the present disclosure can be in real-time and/or based on a predetermined periodicity of at least one of: nanosecond, several nanoseconds, millisecond, several milliseconds, second, several seconds, minute, several minutes, hourly, several hours, daily, several days, weekly, monthly, etc.

The material disclosed herein may be implemented in software or firmware or a combination of them or as instructions stored on a machine-readable medium, which may be read and executed by one or more processors. A machine-readable medium may include any medium and/or mechanism for storing or transmitting information in a form readable by a machine (e.g., a computing device). For example, a machine-readable medium may include read only memory (ROM); random access memory (RAM); magnetic disk storage media; optical storage media; flash memory devices; electrical, optical, acoustical or other forms of propagated signals (e.g., carrier waves, infrared signals, digital signals, etc.), and others.

As used herein, the terms “computer engine” and “engine” identify at least one software component and/or a combination of at least one software component and at least one hardware component which are designed/programmed/configured to manage/control other software and/or hardware components (such as the libraries, software development kits (SDKs), objects, etc.).

Examples of hardware elements may include processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

Computer-related systems, computer systems, and systems, as used herein, include any combination of hardware and software. Examples of software may include software components, programs, applications, operating system software, middleware, firmware, software modules, routines, subroutines, functions, methods, procedures, software interfaces, application program interfaces (API), instruction sets, computer code, computer code segments, words, values, symbols, or any combination thereof. Determining whether an embodiment is implemented using hardware elements and/or software elements may vary in accordance with any number of factors, such as desired computational rate, power levels, heat tolerances, processing cycle budget, input data rates, output data rates, memory resources, data bus speeds and other design or performance constraints.

One or more aspects of at least one embodiment may be implemented by representative instructions stored on a machine-readable medium which represents various logic within the processor, which when read by a machine causes the machine to fabricate logic to perform the techniques described herein. Such representations, known as “IP cores” may be stored on a tangible, machine readable medium and supplied to various customers or manufacturing facilities to load into the fabrication machines that make the logic or processor. Of note, various embodiments described herein may, of course, be implemented using any appropriate hardware and/or computing software languages (e.g., C++, Objective-C, Swift, Java, JavaScript, Python, Perl, QT, etc.).

In some embodiments, one or more of illustrative computer-based systems or platforms of the present disclosure may include or be incorporated, partially or entirely into at least one personal computer (PC), laptop computer, ultra-laptop computer, tablet, touch pad, portable computer, handheld computer, palmtop computer, personal digital assistant (PDA), cellular telephone, combination cellular telephone/PDA, television, smart device (e.g., smart phone, smart tablet or smart television), mobile internet device (MID), messaging device, data communication device, and so forth.

As used herein, term “server” should be understood to refer to a service point which provides processing, database, and communication facilities. By way of example, and not limitation, the term “server” can refer to a single, physical processor with associated communications and data storage and database facilities, or it can refer to a networked or clustered complex of processors and associated network and storage devices, as well as operating software and one or more database systems and application software that support the services provided by the server. Cloud servers are examples.

In some embodiments, as detailed herein, one or more of the computer-based systems of the present disclosure may obtain, manipulate, transfer, store, transform, generate, and/or output any digital object and/or data unit (e.g., from inside and/or outside of a particular application) that can be in any suitable form such as, without limitation, a file, a contact, a task, an email, a message, a map, an entire application (e.g., a calculator), data points, and other suitable data. In some embodiments, as detailed herein, one or more of the computer-based systems of the present disclosure may be implemented across one or more of various computer platforms such as, but not limited to: (1) Linux, (2) Microsoft Windows, (3) OS X (Mac OS), (4) Solaris, (5) UNIX (6) VMWare, (7) Android, (8) Java Platforms, (9) Open Web Platform, (10) Kubernetes or other suitable computer platforms. In some embodiments, illustrative computer-based systems or platforms of the present disclosure may be configured to utilize hardwired circuitry that may be used in place of or in combination with software instructions to implement features consistent with principles of the disclosure. Thus, implementations consistent with principles of the disclosure are not limited to any specific combination of hardware circuitry and software. For example, various embodiments may be embodied in many different ways as a software component such as, without limitation, a stand-alone software package, a combination of software packages, or it may be a software package incorporated as a “tool” in a larger software product.

For example, exemplary software specifically programmed in accordance with one or more principles of the present disclosure may be downloadable from a network, for example, a website, as a stand-alone product or as an add-in package for installation in an existing software application. For example, exemplary software specifically programmed in accordance with one or more principles of the present disclosure may also be available as a client-server software application, or as a web-enabled software application. For example, exemplary software specifically programmed in accordance with one or more principles of the present disclosure may also be embodied as a software package installed on a hardware device.

In some embodiments, illustrative computer-based systems or platforms of the present disclosure may be configured to handle numerous concurrent users that may be, but is not limited to, at least 100 (e.g., but not limited to, 100-999), at least 1,000 (e.g., but not limited to, 1,000-9,999), at least 10,000 (e.g., but not limited to, 10,000-99,999), at least 100,000 (e.g., but not limited to, 100,000-999,999), at least 1,000,000 (e.g., but not limited to, 1,000,000-9,999,999), at least 10,000,000 (e.g., but not limited to, 10,000,000-99,999,999), at least 100,000,000 (e.g., but not limited to, 100,000,000-999,999,999), at least 1,000,000,000 (e.g., but not limited to, 1,000,000,000-999,999,999,999), and so on.

In some embodiments, illustrative computer-based systems or platforms of the present disclosure may be configured to output distinct, specifically programmed graphical user interface implementations of the present disclosure (e.g., a desktop, a web app., etc.). In various implementations of the present disclosure, a final output may be displayed on a displaying screen which may be, without limitation, a screen of a computer, a screen of a mobile device, or the like. In various implementations, the display may be a holographic display. In various implementations, the display may be a transparent surface that may receive a visual projection. Such projections may convey various forms of information, images, or objects. For example, such projections may be a visual overlay for a mobile augmented reality (MAR) application.

In some embodiments, illustrative computer-based systems or platforms of the present disclosure may be configured to be utilized in various applications which may include, but not limited to, gaming, mobile-device games, video chats, video conferences, live video streaming, video streaming and/or augmented reality applications, mobile-device messenger applications, and others similarly suitable computer-device applications.

As used herein, the term “mobile electronic device,” or the like, may refer to any portable electronic device that may or may not be enabled with location tracking functionality (e.g., MAC address, Internet Protocol (IP) address, or the like). For example, a mobile electronic device can include, but is not limited to, a mobile phone, Personal Digital Assistant (PDA), Blackberry™, Pager, Smartphone, or any other reasonable mobile electronic device.

As used herein, terms “cloud,” “Internet cloud,” “cloud computing,” “cloud architecture,” and similar terms correspond to at least one of the following: (1) a large number of computers connected through a real-time communication network (e.g., Internet); (2) providing the ability to run a program or application on many connected computers (e.g., physical machines, virtual machines (VMs)) at the same time; (3) network-based services, which appear to be provided by real server hardware, and are in fact served up by virtual hardware (e.g., virtual servers), simulated by software running on one or more real machines (e.g., allowing to be moved around and scaled up (or down) on the fly without affecting the end user).

In some embodiments, the illustrative computer-based systems or platforms of the present disclosure may be configured to securely store and/or transmit data by utilizing one or more of encryption techniques (e.g., private/public key pair, Triple Data Encryption Standard (3DES), block cipher algorithms (e.g., IDEA, RC2, RCS, CAST and Skipjack), cryptographic hash algorithms (e.g., MD5, RIPEMD-160, RTRO, SHA-1, SHA-2, Tiger (TTH), WHIRLPOOL, RNGs).

The aforementioned examples are, of course, illustrative and not restrictive.

As used herein, the term “user” shall have a meaning of at least one user. In some embodiments, the terms “user”, “subscriber” “consumer” or “customer” should be understood to refer to a user of an application or applications as described herein and/or a consumer of data supplied by a data provider. By way of example, and not limitation, the terms “user” or “subscriber” can refer to a person who receives data provided by the data or service provider over the Internet in a browser session, or can refer to an automated software application which receives the data and stores or processes the data.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A method comprising:
receiving, by at least one processor, a tissue image comprising a digital representation of a plurality of cells of a tissue;
utilizing, by the at least one processor, a histopathological score prediction model to predict at least one mask delineating cells in the tissue image according to cell staining based on learned histopathological scoring parameters;
wherein each mask of the at least one mask is associated with each image band of at least one image band;
wherein each mask of the at least one mask comprises pixel values representative of each image band of the at least one image band;
wherein each image band of the at least one image band represents a cell type classification of at least one cell type classification;
wherein the pixel values of each mask comprises a Gaussian distribution of pixel values centered at each cell of each cell type classification of the at least one cell type classification;
determining, by the at least one processor, a sum of the Gaussian distribution of the pixel values of each mask of the at least one mask;
wherein the sum of the Gaussian distribution of the pixel values of each mask represents a count of cells of each cell type classification;
determining, by the at least one processor, a histopathological score based at least in part on the count of cells of each cell type classification; and
causing to display, by at least one processor, the histopathological score on at least one screen of at least one computing device associated with at least one user.

2. The method as recited in claim 1, wherein the histopathological score prediction model comprises a generative adversarial network (GAN).

3. The method as recited in claim 1, wherein the at least one image band comprises a plurality of grayscale bands.

4. The method as recited in claim 1, further comprising determining, by the at least one processor, a cell-type-specific histopathological score for a particular cell type classification of the at least one cell types classification based at least in part on a ratio of the sum of a mask associated with the particular cell type classification to a total sum of at least one mask.

5. The method as recited in claim 1, further comprising:
receiving, by the at least one processor, an expert annotated tissue sample image comprising a plurality of cell type classification annotations marking a center of each cell of a plurality of cells of a particular cell type classification;
converting, by the at least one processor, the plurality of cell type classification annotations to a training Gaussian mask representing a plurality of true cell type classifications by applying a bivariate normal function having a parameter centered as each cell of the plurality of cells according to the plurality of cell type classification annotations; and
training, by at least one processor, the histopathological score prediction model on the training Gaussian mask.

6. The method as recited in claim 1, wherein a sum of the Gaussian distribution of pixel values centered at each cell of each cell type classification is equal to 1.

7. The method as recited in claim 1, wherein the histopathological score prediction model comprises a Gaussian function to define the Gaussian distribution of the pixel values centered at each cell of each cell type classification of the at least one cell type classification;
wherein Gaussian function comprises parameters for expected value and variance that are customized for a size of cells of each cell type classification.

8. A system comprising:
at least one processor in communication with at least one memory and configured to access instructions stored in the memory that cause the at least one processor to perform steps to:
receive a tissue image comprising a digital representation of a plurality of cells of a tissue;
utilize a histopathological score prediction model to predict at least one mask delineating cells in the tissue image according to cell staining based on learned histopathological scoring parameters;
wherein each mask of the at least one mask is associated with each image band of at least one image band;
wherein each mask of the at least one mask comprises pixel values representative of each image band of the at least one image band;
wherein each image band of the at least one image band represents a cell type classification of at least one cell type classification;
wherein the pixel values of each mask comprises a Gaussian distribution of pixel values centered at each cell of each cell type classification of the at least one cell type classification;
determine a sum of the Gaussian distribution of the pixel values of each mask of the at least one mask;
wherein the sum of the Gaussian distribution of the pixel values of each mask represents a count of cells of each cell type classification;
determine a histopathological score based at least in part on the count of cells of each cell type classification; and
cause to display the histopathological score on at least one screen of at least one computing device associated with at least one user.

9. The system as recited in claim 8, wherein the histopathological score prediction model comprises a generative adversarial network (GAN).

10. The system as recited in claim 8, wherein the at least one image band comprises a plurality of grayscale bands.

11. The system as recited in claim 8, wherein the instructions further cause the at least one processor to perform steps to determine a cell-type-specific histopathological scoring for a particular cell type classification of the at least one cell type classification based at least in part on a ratio of the sum of a mask associated with the particular cell type to a total sum of at least one mask.

12. The system as recited in claim 8, wherein the instructions further cause the at least one processor to perform steps to:
receive an expert annotated tissue sample image comprising a plurality of cell type classification annotations marking a center of each cell of a plurality of cells of a particular cell type classification;
convert the plurality of cell type classification annotations to a training Gaussian mask representing a plurality of true cell type classifications by applying a bivariate normal function having a parameter centered as each cell of the plurality of cells according to the plurality of cell type classification annotations; and
train the histopathological score prediction model on the training Gaussian mask.

13. The system as recited in claim 8, wherein a sum of the Gaussian distribution of pixel values centered at each cell of each cell type classification is equal to 1.

14. The system as recited in claim 8, wherein the histopathological score prediction model comprises a Gaussian function to define the Gaussian distribution of the pixel values centered at each cell of each cell type classification of the at least one cell type classification;
wherein Gaussian function comprises parameters for expected value and variance that are customized for a size of cells of each cell type classification.

15. A non-transitory computer readable medium having software instructions stored thereon, the software instructions configured to cause at least one processor to perform steps comprising:
receiving a tissue image comprising a digital representation of a plurality of cells of a tissue;
utilizing a histopathological score prediction model to predict at least one mask delineating cells in the tissue image according to cell staining based on learned histopathological scoring parameters;
wherein each mask of the at least one mask is associated with each image band of at least one image band;
wherein each mask of the at least one mask comprises pixel values representative of each image band of the at least one image band;
wherein each image band of the at least one image band represents a cell type classification of at least one cell type classification;
wherein the pixel values of each mask comprises a Gaussian distribution of pixel values centered at each cell of each cell type classification of the at least one cell type classification;
determine a sum of the Gaussian distribution of the pixel values of each mask of the at least one mask;
wherein the sum of the Gaussian distribution of the pixel values of each mask represents a count of cells of each cell type classification;
determining a histopathological score based at least in part on the count of cells of each cell type classification; and
causing to display the histopathological score on at least one screen of at least one computing device associated with at least one user.

16. The non-transitory computer readable medium as recited in claim 15, wherein the histopathological score prediction model comprises a generative adversarial network (GAN).

17. The non-transitory computer readable medium as recited in claim 15, wherein the at least one image band comprises a plurality of grayscale bands.

18. The non-transitory computer readable medium as recited in claim 15, wherein the software instructions are further configured to cause the at least one processor to perform steps comprising determining, by the at least one processor, a cell-type-specific histopathological score for a particular cell type classification of the at least one cell type classification based at least in part on a ratio of the sum of a mask associated with the particular cell type to a total sum of at least one mask.

19. The non-transitory computer readable medium as recited in claim 15, wherein the software instructions are further configured to cause the at least one processor to perform steps comprising:
receiving, by the at least one processor, an expert annotated tissue sample image comprising a plurality of cell type classification annotations marking a center of each cell of a plurality of cells of a particular cell type classification;
converting, by the at least one processor, the plurality of cell type classification annotations to a training Gaussian mask representing a plurality of true cell type classifications by applying a bivariate normal function having a parameter centered as each cell of the plurality of cells according to the plurality of cell type classification annotations; and
training, by at least one processor, the histopathological score prediction model on the training Gaussian mask.

20. The non-transitory computer readable medium as recited in claim 15, wherein a sum of the Gaussian distribution of pixel values centered at each cell of each cell type classification is equal to 1.
